(19) United States
(12) Patent Application Publication
(10) Pub. No.: US 2008/0206322 A1
(43) Pub. Date: Aug. 28, 2008

(54) Pharmaceutical Multiparticulate Composit Ion Comprising Mycophenolic Acid or Myco Phenolate Sodium and Combination Compositions with Rapamycin

(75) Inventors: Dieter Becker, Freiburg (DE); Jutta Beyer, Basel (CH); Janez Kerc, Ljubljana (SI); Andrea Kramer, Reute (DE); Nicoletta Loggia, Basel (CH); Chirstian-Peter Luftensteiner, Basel (CH); Jorg Ogorka, Steinen (DE)

Correspondence Address: NOVARTIS;CORPORATE INTELLECTUAL PROPERTY, ONE HEALTH PLAZA 104/3, EAST HANOVER, NJ, 07936-1080, US

(73) Assignee: NOVARTIS AG, Basel (CH)

(21) Appl. No.: 11/573,647

(22) PCT Filed: Aug. 29, 2005
(86) PCT No.: PCT/EP05/09295

§ 371 Date: Jun. 13, 2007

(30) Foreign Application Priority Data
Aug. 31, 2004 (GB) 0419355.3

(51) Int. Cl. A61K 9/52; C07D 307/77; A61K 9/14; A61K 31/4353; A61P 37/06; A61K 31/343; A61K 9/22

(52) U.S. Cl. 424/457; 549/466; 424/468; 424/490; 514/470; 424/497; 424/499; 514/291

(57) ABSTRACT

The present invention relates to a novel composition, e.g. of mycophenolic acid, a salt or a prodrug thereof, in a modified release form.

The present invention relates to a novel composition of mycophenolic acid, a salt or a prodrug thereof.

Mycophenolic acid, also referred to herein as MPA, was first isolated in 1896, and is known to have e.g. anti-tumor, anti-viral, immunosuppressive, anti-psoriatic, and anti-inflammatory activity.

Mycophenolate salts when adapted to be released in the upper part of the intestines lead to effective, well-tolerated, pharmaceuticals particularly for immuno-suppressive indications, e.g. treatment or prevention of cell, tissue or organ allograft rejection. However, there is still a need to further reduce the side-effects of MPA in the gut and reduce variability of drug exposure in the body, e.g. by improving the drug distribution in the intestine or by modifying the drug release profile of the formulation. Furthermore, there is still a need to reduce inter- and intra-patient variability as well as food effect.

Despite mycophenolic acid and mycophenolate salt formulations being already known, there still exists a need for commercially acceptable dosage forms for oral administration with good patient convenience and acceptance.

In accordance with the present invention it has now surprisingly been found that particularly suitable pharmaceutical compositions comprising mycophenolic acid or mycophenolate salt having particularly interesting bioavailability characteristics, being well-tolerated, stable, convenient to administer and with increased swallowability, are obtainable when the compositions are formulated in a modified release form, preferably when the drug substance or a core containing the drug substance is coated with a modified release coating.

Accordingly, the present invention provides:
1. A composition comprising MPA, a salt, e.g. sodium salt, or a prodrug thereof, e.g. MMF, in a modified release form.

As herein defined the composition of the invention comprises MPA, a salt, e.g. sodium salt, or a prodrug thereof, e.g. MMF, in a modified release form.

As herein defined, the wording “salts” encompasses salts, polymorphs, solvates, hydrates or all suitable combinations thereof. Preferred is sodium mycophenolate salt.

Suitable MPA salts include cationic salts, e.g. alkali metal salts, especially the sodium salt, e.g. mono or di-sodium salt, preferably mono-sodium salt.

Prodrugs of MPA include e.g. physiologically hydrolysable esters of MPA, e.g. as disclosed in U.S. Pat. No. 4,753,935 such as the morpholinoethyl ester, also known as mycophenolate mofetil (MMF).

By modified release form is meant a formulation which releases the drug not immediately, e.g. after disintegration or in case of enteric-coating, i.e. gastro-resistant coating, after stomach passage, but offers a sustained, retard, continuous, gradual, prolonged or pulsatile release and therefore alters drug plasma levels distinctively versus an immediate release formulation. More specifically, the term “modified release formulation” as used herein refers to a formulation wherein the active agent is released and provided for absorption over a longer period of time than from a conventional dosage form, i.e. to a formulation which provides a modified release profile of the active agent contained therein

Such a modified release form may be produced by applying release-modifying coatings, e.g. a diffusion coating, to the drug substance or to a core containing the drug substance. Typically these modified release forms provide numerous benefits compared with immediate-release forms including reduced side-effects, greater convenience and higher levels of patient compliance due to a simplified dosing schedule.

The composition of the invention may be e.g. in the form of a tablet or capsule or in a multiparticulate form.

By multiparticles is meant drug particles having an average size of lower than about 3 mm, preferably between about 1 μm to 3 mm. By “average particle size” it is meant that at least 50% of the particulates have a particle size of less than about the given value, by weight. The particle size may be determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

The multiparticulates may be multiparticles, microparticles, minitablets, pellets, granules, beads or drug particles with a modified release coating.

The composition of the invention may comprise a mixture of multiparticulates which provide different modified release profiles, e.g. which comprise different modified release coatings.

The composition of the invention may be a modified release coated, e.g. diffusion coated, tablet or capsule. When the composition of the invention is in the form of a tablet or capsule, it is preferably a tablet or capsule which is able to disintegrate or dissolve to give, e.g. to liberate, multiparticles, e.g. modified release coated multiparticles, e.g. it is preferably a disintegrating tablet or capsule. The tablet or capsule may disintegrate or dissolve in the mouth, stomach or small intestine. The tablet or capsule may release the multiparticles with intact modified release coating.

Preferably the composition of the invention is in a modified release coated multiparticulate form.

When the composition of the invention is in the form of minitablets, it is preferably filled into capsules or aluminium stickpacks, which may provide a high variability of administered doses with the same formulation.

It has been surprisingly found that the compositions of the present invention exhibit especially advantageous properties when administered orally, e.g. in terms of the consistency of pharmacokinetic behavior achieved as indicated in standard bioavailability trials e.g. in healthy subjects. In particular the compositions of the invention provide an improved oral administration form for mycophenolic acid, salt or prodrug thereof, as it exhibits less food interaction, especially with fat rich food. In addition, the variation in mycophenolic acid (MPA) exposure from one day to the next or from day time to night time may be significantly reduced by administering the composition of the invention. Furthermore a better correlation between the trough MPA plasma levels and the total AUC per dose may be reached. Thus with the composition of the invention the pharmacokinetic parameters become more predictable.

According to a further embodiment of the invention, there is provided:
2. Use of a composition of the invention to improve the drug distribution in the intestine, to delay the delivery of the drug substance to the intestinal tract, to reduce inter- and intra-patient variability, to reduce or prevent food effect or GI effects, to increase swallowability or increase patient compliance.
3. A method for improving the drug distribution in the intestine, delaying the delivery of the drug substance to the intestinal tract, reducing inter- and intra-patient PK variability, or reducing or preventing food effect in a subject, e.g. a transplanted subject or a subject having an autoimmune disease, comprising administering a therapeutically effective amount of a composition of the invention.
4. A method for treating and/or preventing native or transgenic organ, tissue or cellular allograft or xenograft transplant rejection, or immune-mediated and/or inflammatory disease, which comprises administering a therapeutically effective amount of a composition of the invention in a subject in need thereof, optionally with the simultaneous, sequential or separate administration of another immunosuppressant.
5. Use of a composition of the invention to improve the drug distribution in the intestine, to delay the delivery of the drug substance to the intestinal tract, to reduce inter- and intra-patient PK variability, to reduce or prevent food effect or GI effects, to increase swallowability or increase patient compliance.
6. Use of a composition of the invention in the manufacture of a medicament for the treatment and/or prevention of native or transgenic organ, tissue or cellular allograft or xenograft transplant rejection, or immune-mediated and/or inflammatory disease.

The composition with modified release according to the invention may conveniently be coated with a component which offers a sustained, continuous, gradual, prolonged or pulsatile release of MPA, MPA salt or MPA prodrug in the body, preferably in the intestine, e.g. a modified release coating, e.g. a diffusion coating.

Examples of such modified release coating components are e.g. cellulose derivatives; e.g. ethylcellulose, e.g. Aquacoat® ECD, available from FMC; Surelease available from Colorcon, acrylic copolymers, preferably acrylic and methacrylic copolymers containing quaternary ammonium groups, e.g. tri(C₁₋₄alkyl)-ammonium methylmethacrylate groups, e.g. trimethylammonium methylmethacrylate groups, e.g. acrylic/methacrylicacid-ester with different ratio of quarternary ammonium groups 20:1 RL/40:1 RS, e.g. such polymers commercially available from Röhm Pharma under the Trademarks, Eudragit RL^{R}, Eudragit RS^{R} or Eudragit NE^{R} or copolymers; and/or mixtures thereof. A ratio of about 75:25, preferably 90:10, preferably 95:5 by weight Eudragit RS^{R}:Eudragit RL^{R} is particularly preferred.

The modified release coating components may be in aqueous dispersion, e.g. as 30% aqueous dispersion, or organic solution, e.g. 12.5% organic solution. For example the modified release coating components is a mixture of Eudragit RL^{R} and Eudragit RS^{R} in 30% aqueous dispersion or 12.5% organic solution.

The amount of modified release coating components may be from about 30 to about 100 weight %, more preferably from about 50 to about 100 weight %, based on the total weight of the coating.

The modified release coating, e.g. diffusion coating, preferably comprises 5 to 50 weight %, more preferably 5-20 weight %, even more preferably 10-15 weight %, of the total weight of the composition.

The skilled person would adjust the nature and amount of modified release coating polymer to adjust as necessary the profile release of the MPA, salt or prodrug thereof, containing in the composition of the invention.

The modified release coating may further include one or more further components or excipiens, e.g. pore formers, a plasticizer, an antisticking agent, a wetting agent, e.g. as disclosed hereinafter.

In another aspect of the invention, there is provided
7. A composition comprising a drug, e.g. an immunosuppressant, e.g. MPA, a salt or a prodrug thereof, e.g. MMF, containing a modified release coating, e.g. a diffusion coating, wherein the modified release coating contains a pore former, e.g. an enteric pore-former, e.g. a pH dependent pore-former, e.g. as hereinabove defined.
8. Use of such a composition to improve the drug distribution in the intestine, to delay the delivery of the drug substance to the intestinal tract, to reduce inter- and intra-patient PK variability, to reduce or prevent food effect or GI effects, to increase swallowability or increase patient compliance.
9. Method for treating and/or preventing native or transgenic organ, tissue or cellular allograft or xenograft transplant rejection, or immune-mediated and/or inflammatory disease, which comprises administering such a composition in a subject in need thereof, optionally with the simultaneous, sequential or separate administration of another immunosuppressant.
10. Use of such a composition in the manufacture of a medicament for the treatment and/or prevention of native or transgenic organ, tissue or cellular allograft or xenograft transplant rejection, or immune-mediated and/or inflammatory disease

Suitable pore-formers may be pH independent pore-formers, such as HPMC, or pore-formers which are pH dependent, Suitable pH dependent pore-formers may be enteric pore-formers, e.g. enteric coating polymers.

As herein defined, an enteric pore-former is a pore-former which provides drug release in an environment with pH>5, e.g. in intestinal fluid, and suppresses drug release in acidic environment, e.g. in the stomach. Example of enteric pore-formers according to the present invention are HPMC-phthalate (HPMC-P), e.g. HP50, HP55, e.g. from ShinEtsu; HPMC-acetate-succinate (HPMC-AS), e.g. Aqoat LF or Aqoat MF, e.g. from ShinEtsu; Methyl acrylic acid-ethyl acylic acid copolymer, e.g. Methacrylic acid copolymer, e.g. Eudragit L, S, L100-55 and/or L30D from Röhm Pharma, Acryl-Eze from Colorcon, Kollicoat MAE 30 DP from BASF; Celluloseacetatephthalate, e.g. Aquacoat CPD from FMC Biopolymer, or Polymer from Eastman Kodak; and Polyvinylacetatephthalate, e.g. Sureteric, Colorcon, or any mixture thereof. Preferably HPMC-P and HPMC-AS may be combined with ethylcellulose or acrylic and methacrylic copolymers containing quaternary ammonium groups, e.g. tri(C₁₋₄alkyl)-ammonium methylmethacrylate groups, e.g. Eudragit RS in organic coating solutions, HPMC-AS dispersed in water can also be combined with aqueous ethylcellulose dispersion e.g. Aquacoat ECD, FMC.

It has been surprisingly shown that in the case of MPA, which has a poor solubility in acidic medium, the enteric pore formers advantageously reduce the effect of acidic pH pretreatement on drug release compared to water soluble pore formers.

Hydroxypropyl methylcellulose phthalates, typically have a molecular weight of from 20,000 to 100,000 Daltons e.g. 80,000 to 130,000 Daltons, e.g. a hydroxypropyl content of from 5 to 10%, a methoxy content of from 18 to 24% and a phthalyl content from 21 to 35%. Examples of suitable hydroxypropyl methylcellulose phthalates are the marketed products having a hydroxypropyl content of from 6-10%, a methoxy content of from 20-24%, a phthalyl content of from 21-27%, a molecular weight of about 84,000 Daltons known under the trade mark HP50 and available from Shin-Etsu Chemical Co. Ltd., Tokyo, Japan, and having a hydroxypropyl content, a methoxy content, and a phthalyl content of 5-9%, 18-22% and 27-35% respectively, and a molecular weight of 78,000 Daltons, known under the trademark HP55 and available from the same supplier.

Examples of suitable hydroxypropylmethylcellulose acetate succinate may be used as known under the trademark Aqoat LF or Aqoat MF and commercially available, e.g. from Shin-Etsu Chemical Co. Ltd., Tokyo, Japan.

The modified release coating of the composition of the invention may comprise 0 to 70 weight %, more preferably 5 to 50 weight % of pore-former, based on the total weight of the modified release coating.

The composition of the invention may further include a pore-former, e.g. which gives water-soluble pores, e.g. polyethyleneglycol, polyvinylpyrrolidone, polyethylene oxide, a cellulose derivative, e.g. hydroxyethyl cellulose, Hydroxypropylmethylcellulose (HPMC), Hydroxypropylcellulose, or other cellulose derivatives, e.g. which are soluble in acidic medium, e.g. as ammonium salt, acrylate or methacrylate esters, e.g. Eudragit E or Eudragit EPO; polyacrylic acid; which are swelling in water, e.g. Eudragit RS, RL, NE 30D, which are soluble in alkaline medium, i.e. enteric coating polymer, e.g. Eudragit L, S, L100-55 or any mixture thereof. HPMC may also act as a thickening agent due to the viscosity of the aqueous solution thereof. According to the invention the pore formers may be hydrophilic agents, e.g. water soluble platisizers, e.g. PEG, triacetine, triethylcitrate, or hydrophilic silicium dioxide, e.g. Aerosil 200 or Syloid 244 FP.

Suitable plasticizers according to the invention include e.g., triacetine, triethy citrate, tributyl citrate, dibutylsebacate, diethyl sebacate, polyethyleneglycol 400, 3000, 4000 or 6000, acetyltriethylcitrate, acetyltributylcitrate, and diethylphthalate, or mixtures thereof. Preferably the plasitcizer is triethylcitrate or dibutylsebacate A plasticizer generally swells the coating polymer such that the polymer's glass transition temperature is lowered, its flexibility and toughness increased and its permeability altered. When the plasticizer is hydrophilic, such as polyethylene glycol, the water permeability of the coating is generally increased. When the plasticizer is hydrophobic, such as diethyl phthalate or dibutyl sebacate, the water permeability of the coating is generally decreased.

Preferably the plasticizer is present in an amount of 1 to 50% by weight, preferably 2 to 35%, more preferable 5-25% based on the total weight of the coating.

Examples of antisticking agents are silicon dioxide, e.g. colloidal silicon dioxide, an synthetic amorphous silicic acid such as Syloid 244 FP, talc, Aerosil 200 or glycerine monostearate.

Preferably the antisticking agent is Areosil 200 and Syloid 244 FP. When the antisticking agent is hydrophilic, such as Aerosil 200 or Syloid 244 FP, the water permeability/swelling (and therefore also drug release) of the coating is generally increased. When the plasticizer is hydrophobic, such as talcum or glycerolmonostearate, the water permeability of the coating is generally decreased. Antisticking agents are optionally included in the coating formulation to avoid sticking of the drug cores and guarantee a high separation of them.

Preferably the antisticking agent is present in an amount of 1 to 50% by weight, more preferably 5 to 25% by weight, based on the total weight of the coating.

Suitable wetting agents include e.g. sodium laurylsulphate, cetomacrogol, a wax, glycerol monostearate, a sorbitan ester and a poloxamer. Wetting agents are optionally included in the coating formulation due to their property to reduce interfacial tensions and improve the contact of spray solutions or suspensions with treated surfaces.

Preferably the wetting agent is present in an amount of 1 to 20% by weight, more preferably 1 to 5% by weight, based on the weight of the coating.

The composition of the invention may be additionally enteric coated. By enteric coated or coating is meant a pharmaceutically acceptable coating preventing the release of the active agent in the stomach and allowing the release in the upper part of the intestinal tract. The enteric coating may be added as an overcoat upon the modified release coating.

The preferred enteric coating for the composition of the invention comprises a film-forming agent selected from e.g. cellulose acetate phthalate; cellulose acetate trimellitate; methacrylic acid copolymers, e.g. copolymers derived from methylacrylic acid and esters thereof, containing at least 40% methylacrylic acid; hydroxypropyl methylcellulose phthalate; hydroxypropylmethylcellulose acetate succinate or Polyvinylacetatephthalate,

Typical cellulose acetate phthalates have an acetyl content of 17-26% and a phthalate content of from 30-40% with a viscosity of ca. 45-90 cP. An example of an appropriate cellulose acetate phthalate is the marketed product CAP (Eastman Kodak, Rochester N.Y., USA or Aquacoat CPD from FMC Biopolymer).

Typical cellulose acetate trimellitates have an acetyl content of 17-26%, a trimellityl content from 25-35% with a viscosity of ca. 15-20 cS. An example of an appropriate cellulose acetate trimellitate is the marketed product CAT (Eastman Kodak Company, USA).

Methacryclic acid copolymers include preferably copolymers derived from methylacrylic acid and esters thereof, containing at least 40% methylacrylic acid, more preferably those of molecular weight above 100,000 Daltons based on, e.g. methylacrylate and methyl or ethyl methylacrylate in a ratio of about 1:1. Typical products include Eudragit L, e.g. L 100-55, L30 D marketed by Rohm GmbH, Darmstadt, Germany or Acryl-Eze from Colorcon, Kollicoat MAE 30 DP from BASF.

HPMC-phthalates and HPMC-acetate succinate are as defined hereinabove. Examples of suitable HPMC-phthalates are HP50 or HP55. Examples of suitable hydroxypropylmethylcellulose acetate succinate may be used as known under the trademark Aqoat LF or Aqoat MF (both Shin-Etsu).

The enteric coating may further comprise further components such as a plasticizer, e.g. triacetine, triethylcitrate, diethylsebacate, polyethyleneglycol 3000, 4000 or 6000, acetyltriethylcitrate, acetyltributylcitrate, or diethylphthalate, and/or antisticking agents, e.g. colloidal silicon dioxide, an synthetic amorphous silicic acid such as Syloid 244 FP, talc, or glycerine monostearate. The coating may further comprise, especially in aqueous dispersions, one or more thickening agents to avoid sedimentation of suspended excipients, e.g. HPMC 3 cps or HPMC 6 cps.

Preferably the enteric-coating may further comprise a film-forming agent, e.g. cellulose acetate phthalate, cellulose acetate trimellitate, methacrylic acid copolymer, hydroxypropyl methylcellulose phthalate or hydroxypropylmethylcellulose acetate succinate, polyvinylacetatephthalate. The amount of the film-forming agent may be from 50 to 95% by weight, based on the total weight of the enteric coating, more preferably 60 to 80% by weight. The plasticizer and/or the antisticking agent, if present in the enteric-coating, may be e.g. as disclosed above for the modified release coat, e.g. in the amount as indicated above for the modified release coat.

According to the invention, the drug substance is preferably present in the composition of the invention in an amount of 1 to 99% by weight, based on the total weight of the core (i.e. excluding the coating). In particular when the composition of the invention is in the form of, small tablets, minitablets, pellets, beads or granules, the drug substance is preferably present in an amount of 1 to 95% by weight, more preferably 20 to 90%, most preferably 30 to 80% by weight, based on the total weight of the core (i.e. excluding the coating). When the composition of the invention is in the form of particles, or microparticles the drug substance is preferably present in an amount of 1 to 95% by weight, more preferably to 50-95%, most preferably to 70-90% by weight, based on the total weight of the core (i.e. excluding the coating.

The composition of the invention may contain one or more excipients or diluents, e.g. as hereinafter disclosed.

A preferred group of drug microparticles according to the invention are those having an effective average particle size of less than about 1000 μm, preferably between about 10 and 800 μm, more preferably between 30 and 200 μm. The drug microparticles may optionally be combined with one or more pharmaceutically acceptable coating ingredients, e.g. ethylcellulose or a methacrylic acid copolymer, and a stabilizer, e.g. colloidal silica, to form the microparticle drug core, for instance by spray-drying, fluid bed drying or precipitation techniques.

Crystalline mycophenolic acid salt particles, e.g. in a size range between 1 and 200 microm (μm), may also be prepared by means of high pressure homogenization of a suspension of unmilled crystalline drug crystals in any fluid in which the drug substance is sparsely soluble, such as water and organic solvents, e.g. methylene chloride or ethanol/acetone mixtures.

These microparticulate drug suspensions may be directly coated by a polymer layer, or embedded in a polymer matrix, e.g. by adding the polymer and dissolving it in the homogenized suspension which is subsequently spray dried or spray granulated. Preferably polymers used are Ethylcellulose or acrylic and methacrylic copolymers containing quaternary ammonium groups.

The precipitation techniques may also include the coacervation techniques, e.g. to separate a liquid phase of a coating material from a polymeric solution and wrapping of that phase as a uniform layer around suspended core particles. The resulting microparticles may be collected by filtration or centrifugation, washed with an appropriate solvent, and subsequently dried by standard techniques such as spray drying or fluidized bed drying.

The drug particles may then be coated with modified release coating ingredients as disclosed herein, and optionally a stabilizer, e.g. colloidal silica. The modified release coating may be prepared for instance by fluid-bed coating and/or granulation or precipitation techniques.

The resulting coated drug particles may optionally be combined with a diluent, e.g. as disclosed hereinafter, for example lactose, mannitol or sucrose, a lubricant, e.g. as disclosed hereinafter, for instance magnesium stearate, and dispensed in a capsule or a sachet or compressed into tablets.

In another embodiment the drug substance may optionally be combined with a binder or optionally with diluent and a binder, e.g. as disclosed herein after, and formed into granules, e.g. using a technique such as high or low shear granulation or fluid bed granulation to form the granule drug core. The granules obtained may then be coated with modified release coating ingredients, e.g. as disclosed herein, and e.g. dispensed in a capsule or a sachet. The granule drug core typically has a mean width of diameter of from 0.05 to 2 mm or preferably form 0.1 to 2 mm, or more preferably of from 0.15 to 1.5 mm. The amount of drug substance present in the core may be from 1 to 95% or preferably form 20 to 90%, or more preferably from 50 to 90% by weight, based on the total weight of the granule drug core (i.e. excluding the coating).

Drug particles were the drug is in the form of crystals, amorphous particles or a mixture thereof can also be used for subsequent coating.

In another embodiment the drug substance may optionally be combined with one or more pharmaceutically acceptable extrusion aid(s), e.g. microcrystalline cellulose, an amylose pregelled starch, etc., binder(s), e.g. as herein disclosed, or diluents, e.g. as herein disclosed, and formed into pellets, e.g. using a technique such as extrusion spheronisation, direct pelletisation/high or low shear granulation, fluid bed granulation or spray drying/melt concealing, to form the pellet drug core. The pellets obtained may be coated with modified release coating ingredients, e.g. as herein disclosed, and dispensed in a capsule or a sachet. The pellet drug core typically has a width of diameter of from 0.2 to 2 mm, preferably of from 0.5 to 1.4 mm. The amount of drug substance present in the core may be from 1 to 95% by weight, based on the total weight of the pellet drug core (i.e. excluding the coating).

In another embodiment, the drug optionally in combination with a pharmaceutically acceptable binder, may be layered onto the surface of a pharmaceutically acceptable seed, typically a particle (e.g. a sphere) of sucrose, starch, microcrystalline cellulose or any combination thereof, to form the bead drug core. Such layering may be solution layering or powder layering. Such a pharmaceutically acceptable seed is preferably a non-pareil sugar/starch sphere of 18-20 mesh, 25-30 mesh or 35-40 mesh, most preferably a non-pareil sugar starch sphere of 25-30 mesh or Cellets, i.e. microcrystalline cellulose beads e.g. from Pharmatrans Sanaq AG, in the size range of 100-1000 μm, more preferably 100-200 and 200-355 μm. The beads obtained may be coated with modified release coating ingredients, e.g. as herein disclosed, and dispensed in a capsule or a sachet or further processed by layering of another drug. The bead drug core typically has a width of diameter of from 0.2 to 2 mm, preferably of from 0.5 to 1.4 mm. The amount of drug substance present in the core may be from 1 to 95% by weight, based on the total weight of the bead drug core (i.e. excluding the coating).

In a further embodiment, coated drug particle or coated granules or coated pellet drug cores may optionally be combined with pharmaceutically acceptable ingredients, e.g. a diluent, binder, lubricant, e.g. as herein disclosed, well known to the skilled person to form tablets and or small tablets which disintegrate in the stomach and release the coated drug particles, or coated pellets or coated granules.

The term “small tablets” within the scope of this application denotes tablets with an overall size of about 3 to 5 mm.

The term “minitablets” within the scope of this application denotes small tablets with an overall weight of approximately 2 to 30 mg, e.g. approximately 4 to 9 mg, e.g. approximately 7 mg, in their uncoated form. The minitablets may have any shape known to the skilled person for tablets, e.g. round e.g. with a diameter of about 1.5 to 3 mm; cyclindrical e.g. having a convex upper face and convex lower face and e.g. with a cylindrical diameter and height independently of each other are from 1 to 3 mm; or biconvex minitablets e.g. whose height and diameter are approximately equal and are from 1.5 to 3 mm.

Minitablets comprising mycophenolic acid, a salt or a prodrug thereof, e.g. MMF, are preferably of a total weight (i.e. the weight of the tablet core plus the weight of coating) of 3 to 12 mg.

MPA, a salt thereof, or a prodrug thereof, e.g. MMF, may be granulated prior to the preparation of minitablets or small tablets

The tablets consist of the drug granulate, i.e. the drug (MPA, a salt thereof, or a prodrug thereof, e.g. MMF) a binder and a filler. This granulate may be compressed into tablets/minitablets optionally with additional filler, binder, disintegrant and lubricant.

Examples of fillers include e.g. a water-soluble or water-insoluble saccharide such as lactose or mannitol; glucose anhydrate; microcrystalline cellulose, e.g. as known and commercially available under the trade name Avicel® from FMC Corporation; colloidal silicon dioxide, e.g. as known and commercially available under the trade name Aerosil®; or an amylose pre-gelled starch. The composition of the invention preferably comprises the filler in an amount of 10 to 90% by weight, based on the total weight of the uncoated composition, more preferably 10 to 50% by weight, most preferably 15 to 35% by weight.

Examples of binders include e.g. polyvinylpyrrolidone (PVP), e.g. PVP K30 or PVP K12, as known and commercially available under the trade name Povidone® from the BASF company, e.g. Povidone K-30; or hydroxypropylmethylcellulose (HPMC), e.g. HMPC with a low apparent viscosity, e.g. below 100 cps as measured at 20° C. for a 2% by weight aqueous solution, e.g. below 50 cps, preferably below 20 cps, for example HPMC 3 cps, as known and commercially available under the name Pharmacoat® 603 from the Shin-Etsu company; or sodium carboxymethylcellulose. Preferably the composition of the invention comprises the binder in an amount of 1 to 30% by weight, based on the total weight of the uncoated composition, more preferably 1 to 20% by weight, most preferably 5 to 15% by weight.

Examples of disintegrants are e.g. natural starches, such as i) maize starch, potato starch, and the like, ii) directly compressible starches, e.g. Sta-rx® 1500, modified starches, e.g. carboxymethyl starches and sodium starch glycolate, available as Primojel®, Explotab®, Explosol®, and iii) starch derivatives such as ephrit; crosslinked polyvinylpyrrolidones, e.g. crospovidones, e.g. Polyplasdone® XL and Kollidon® CL; alginic acid or sodium alginate; methacrylic acid-divinylbenzene copolymer salts, e.g. Amberlite® IRP-88; and cross-linked sodium carboxymethylcellulose, available as e.g. Ac-di-sol®, Primellose®, Pharmacel® XL, Explocel®, and Nymcel® ZSX, or a mixture thereof. The composition of the invention preferably comprises the disintegrant in an amount of up to 20% by weight, based on the total weight of the uncoated composition, more preferably 0 to 15%.

Preferably, the modified release coated compositions according to the invention, e.g. comprising MPA, a salt or a prodrug thereof, e.g. MMF and optionally an enteric pore forming agent, are free of any disintegrating agent.

Examples of lubricants are e.g. magnesium stearate, hydrogenated castor oil, glycerine monostearate, or sodium fumarylstearate, e.g. in an amount of 0.1 to 3% by weight, based on the total weight of the uncoated composition.

Procedures which may be used to prepare and/or to coating the compositions of the invention may be conventional or known in the art or based on such procedures e.g. those described in L. Lachman et al. The Theory and Practice of Industrial Pharmacy, 3^{rd} Ed, 1986, H. Sucker et al, Pharmazeutische Technologie, Thieme, 1991, Hager's Handbuch der pharmazeutischen Praxis, 4^{th} Ed. (Springer Verlag, 1971) and Remington's Pharmaceutical Sciences, 13^{th} Ed., (Mack Publ., Co., 1970) or later editions. Minitablets may e.g. manufactured on a standard rotary tabletting machine.

The modified release of the compositions of the invention may be analyzed by techniques known by the one skilled in the art, e.g. by defining the dissolution rate profile of the composition, e.g. by determining the amount of dissolved active substance per time unit.

The compositions of the invention are useful as immunosuppressants as indicated by standard tests. The activity and characteristics of the compositions of the invention may be indicated in standard
a) clinical trials, e.g. observing the first acute rejection episodes or treatment failure six months after transplant of kidneys or maintaining a rejection-free state within 6 months after initiation of treatment with the invention. The compositions of the invention are administered at a dose in the range of 0.5 to 2.0 g/day e.g. about 1.5 g/day and decrease the acute rejection rates when administered during the period around transplant surgery, and maintain a rejection-free state in patients who are 3 months or more after transplantation. Thus the compositions of the invention may be administered during the initial 72 hours after transplantation at dose of about 0.5 g administered twice a day in combination with a conventional steroid and cyclosporin, e.g. as NEORAL^{R} for which the cyclosporin dose is the conventional dose e.g. ca 8±3 mg/kg for renal transplants. The steroid dose is to be administered at about 2.5 mg/kg for 4 days after transplant, 1 mg/kg thereafter for 1 week, 0.6 mg/kg thereafter for 2 weeks thereafter 0.3 mg/kg for 1 month for prednisone, and in
b) animal trials e.g. observing the kidney allograft reaction in rat. In this test one kidney from a female fisher 344 rat is transplanted onto the renal vessel of a unilaterally (left side) nephrectomized WF recipient rat using an end-to-end anastomosis. Ureteric ananstomosis is also end-to-end. Treatment commences on the day of transplantation and is continued for 14 days. A contralateral nephrectomy is done seven days after transplantation, leaving the recipient relying on the performance of the donor kidney. Survival of the graft recipient is taken as the parameter for a functional graft. Typical doses of the compositions of the invention are from about 1 to 30 mg/kg p.o.

The compositions of the invention lead to an inter- and intra-patient reduced variability of MPA, MPA salt, for example sodium mycophenolate, or MPA prodrug, for example MMF, and to a beneficial release profile of the drug substance.

The compositions of the invention are particularly useful for the following conditions:
a) Treatment or prevention of native or transgenic organ, tissue or cellular allograft or xenograft transplant rejection, e.g. for the treatment of recipients of e.g. heart, lung, combined heart-lung, liver, kidney, pancreatic, skin, pancreatic islet cell, neural cell or corneal transplant; including treatment and prevention of acute rejection; and treatment and prevention of chronic rejection, e.g. as associated with graft-vessel disease. The compositions of the invention are also indicated for the treatment and prevention of graft-versus-host disease, such as following bone marrow transplantation.
b) Treatment and prevention of autoimmune diseases, e.g. immune-mediated diseases and inflammatory conditions, in particular inflammatory conditions with an etiology including an immunological component such as arthritis (for example rheumatoid arthritis, arthritis chronica progrediente and arthritis deformans) and rheumatic diseases. Specific immune-mediated diseases for which the compositions of the invention may be employed include, autoimmune hematological disorders, including, but not limited to hemolytic anaemia, aplastic anaemia, pure red cell anaemia and idiopathic thrombocytopenia), systemic lupus erythematosus, polychondritis, sclerodoma, Wegener granulosis, dermatomyositis, polymyositis, chronic active hepatitis, primary bilary cirrhosis, myasthenia gravis, psoriasis, Steven-Johnson syndrome, pemphigus, idiophatic sprue, inflammatory bowel diseases (including e.g. ulcerative colitis and Crohn's disease), endocrine ophthalmophathy, Graves disease, sarcoidosis, multiple sclerosis, juvenile diabetes (diabetes mellitus type I), non-infectious uveitis (anterior and posterior), keratoconjunctivitis sicca and vernal keratoconjunctivitis, interstitial lung fibrosis, psoriatic arthritis, vasculitis, glomerulonephritides (with and without ephritic syndrome, e.g. including idiophatic ephritic syndrome or minimal change nephropathy) and juvenile dermatomyositis.

In particular, the present combinations of the invention are useful for the treatment and prevention of acute or chronic rejection, including maintenance patients.

The dose of the MPA, MPA salt, e.g. sodium mycophenolate salt, or MPA prodrug, e.g. MMF, may vary depending on a variety of factors, for example the compound chosen, the particular condition to be treated and the desired effect. In general satisfactory results are obtained on administration e.g. orally at daily dosages on the order of e.g. from about 50 mg to about 2.5 g MPA per day, e.g. about 250 mg to about 2.2 g MPA, e.g. about 360 mg, about 720 mg, about 740 mg, about 1.1 g, about 1.5 g, about 2.2 g, administered as a single dose or in divided doses, preferably about 360 mg to 720 mg MPA twice a day. Dosages of MPA salt or prodrug are to be calculated to correspond to the above mentioned dosages of MPA.

The compositions of the invention may be used the sole active drug or together with other drugs in immunomodulating regimens or other anti-inflammatory agents e.g. for the treatment or prevention of allograft acute or chronic rejection or autoimmune disorders. For example, a manzamine may be used in combination with a calcineurin inhibitor, e.g. cyclosporine or cyclosporine derivatives, e.g. cyclosporine A or cyclosporine G, FK-506, ABT-281, ASM 981; an mTOR inhibitor, e.g. rapamycin or rapamycin derivatives, e.g. 40-O-(2-hydroxy)ethyl-rapamycin, CCI779, ABT578, AP23573, AP23464, AP23675, AP23841, TAFA-93, biolimus-7 or biolimus-9; a corticosteroid; cyclophosphamide; azathioprine; methotrexate; a S1P receptor agonist, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CDS, CD4, CD11a/CD18, CD7, CD25, CD27, B7, CD40, CD45, CD58, CD137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4lg (for ex. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists.

The terms “co-administration” or “combined administration” or the like as utilized herein are meant to encompass administration of the drug substance to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the drug substance and the active co-agent are both administered to a patient simultaneously in the form of a single entity or dosage. The term “non-fixed combination” means that the active ingredients, e.g. a compound of formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

The compositions of the invention may preferably be used to prepare a fixed combination with rapamycin or a derivative thereof, e.g. 40-O-(2-hydroxy)ethyl-rapamycin, CCI779, ABT578, AP23573, AP23464, AP23675, AP23841, TAFA-93, biolimus-7 or biolimus-9. Examples of fixed combinations are e.g. as disclosed in UK patent applications Nos. 323202, 323598, 329852, 405902 and 410714, the contents thereof being incorporated herein by reference, wherein the MPA, sodium mycophenolate or MMF containing sub-units or units are replaced by a composition according to the invention.

The following examples illustrate various aspects of the invention.

### EXAMPLE 1

### Preparation of Granules

### Formulation 1.A

A dry blend is made by mixing the drug, Aerosil 200, Povidone (PVP) K30 and lactose in a planetary or high shear mixer. Ethanol is added to produce granules which are thoroughly dried and sieved for suitable size selection.

| Composition (amounts given in weight %) of the core | % | % | % |
|---|---|---|---|
| MPA, Na Mycophenolate or MMF | 50 | 30 | 60 |
| Povidone K-30 | 5 | 5 | 5 |
| Aerosil 200 | 2 | 2 | 2 |

### Formulation 1.B

The drug substance is mixed with part the binder (ethylcellulose) in a laboratory high shear mixer. The remaining part of the binder is dissolved in the granulation fluid (ethanol). The granulation fluid is added into the mixer continuously till the granulation end point is reached. The granules are sized through a screen to destroy lumps and dried in a fluid-bed dryer. The resulting granules are screened to reach a suitable final granules size.

| Composition (amounts given in weight % of the core): | % |
|---|---|
| Mycophenolate Sodium or MMF | 88.7 |
| Ethylcellulose N7 dry | 9.4 |
| Ethylcellulose N7 (in granulation fluid) | 1.9 |
| Ethanol 96%* | q.s. |

*removed during processing

### Formulation 1.C:

The drug substance is mixed with the binder in a laboratory high shear mixer. The granulation fluid is added into the mixer continuously till the granulation end point is reached. The granules are sized through a screen to destroy lumps and dried in a fluid-bed dryer. The resulting granules are screened to reach a suitable final granules size.

| Composition (amounts given in weight % of the core): | % |
|---|---|
| Mycophenolate Sodium or MMF | 90.5 |
| PVP K30 | 9.5 |
| Ethanol 94%* | q.s. |

*removed during processing

The resulting granules of formulations 1.A, 1.B, 1.C may be coated e.g. with one of the coating formulations 5:A, 5:B, 5.C, 5.D or 6.4 below by using a coating equipment, e.g. a fluid-bed dryer with a Wurster column. Coated drug particles may then be formulated into a capsule or sachet by the addition of bulking agents and lubricants or further compressed into tablets or minitablets.

### EXAMPLE 2

### Preparation of Pellets

A dry blend is made by mixing the drug, microcrystalline cellulose (Avicel PH101) and lactose in a planetary mixer. Purified water is added to give a wet mass that is subsequently extruded using a screen of a suitable size. The extrudates are rounded in a spheroniser, thoroughly dried and sieved for suitable size selection.

The resulting pellets finally are coated with an aqueous dispersion or organic solution of the coating formulations below.

Composition (amounts given in % of the core)

| | | | |
|---|---|---|---|
| MPA, Na Mycophenolate or MMF | 50% | 30% | 60% |
| Lactose (standard grade) | 25% | 35% | 20% |
| Microcrystalline cellulose (Avicel PH1) | 25% | 35% | 20% |
| Water for wet massing | q.s.* | q.s.* | q.s.* |

*removed during processing.

### EXAMPLE 3

### Preparation of Beads

Drug solutions are prepared by dissolving the drug, and the formulation components as described below in the selected media with mixing.

### Formulation 3.A

Non-pareil seeds are dispensed into a Wurster fluid bed coater or in a Hüttlin type of fluidized bed coater and fluidized. The drug solution previously prepared is then sprayed onto the seeds until the drug solution is depleted. The beads are dried in the same conditions for 5 minutes. The beads of formulation 3.A are then finally coated with an aqueous dispersion or an organic solution of the coating ingredients of the coating formulation below and dried for 15 minutes. Beads may then be dispensed in a capsule or sachet. The formulation is to be applied onto 1000 g non-pareil seeds.

(amounts given in %)

| | | | |
|---|---|---|---|
| MPA, Na Mycophenolate or MMF | 80% | 60% | 40% |
| Hydroxypropyl methylcellulose(Methocel E50LV) | 18% | 36% | 54% |
| Polyethylene glycol (PEG 400) | 2% | 4% | 6% |
| Ethanol/Water (70:30) | q.s.* | q.s.* | q.s.* |

*Removed during processing

### Formulation 3.B

Non-pareil seeds are dispensed into a Wurster fluid bed coater or in a Hüttlin type of fluidized bed coater and fluidized. The drug solution previously prepared is then sprayed onto the seeds until the drug solution is depleted. The beads are then sprayed with a solution/suspension of one of the coating formulations 5.A, 5.B or 5.C below and, after drying, with a solution of hydroxypropyl methylcellulose (Opadry) in water and finally dried for 10 minutes. Beads can then dispensed in a capsule or sachet. The formulations is to be applied onto 1000 g non-pareil seeds.

(amounts given in %)

| | | | |
|---|---|---|---|
| Compound A | 80% | 60% | 40% |
| Talc | 8% | 15% | 24% |
| Hydroxypropyl methylcellulose (Opadry) | 12% | 25% | 36% |
| Water | q.s.* | q.s.* | q.s.* |

*Removed during processing.

Beads for formulations 3.A and 3.B may be used as a combination by including them into the same capsule or sachet.

Alternately, beads may also be prepared by combining formulations 3.A and 3.B onto the same non-pareil seeds according to the following process. Formulation 3.A is firstly sprayed onto the beads, followed by one of the coating formulations below and finally formulation 3.B.

The resulting layered beads are finally coated with an aqueous dispersion or organic solution of the coating formulations below.

### EXAMPLE 4

### Preparation of Minitablets (Small Tablets)

Minitablets of sodium mycophenolate are prepared by granulation of sodium mycophenolate, Aerosil 200 and Povidone (PVP) K30 with ethanol 94% for granulation in an amount as indicated in Tables 1-3. After grinding, drying and sieving, the granulate is mixed with the other ingredients as given in Tables 1-3 at dry stage and compressed into minitablets. To give modified release tablets the minitablet formulation does not contain disintegrants in most examples

TABLE 1
Compositions of a minitablet of sodium mycophenolate (amounts given
in mg)

| Core | 4.A | 4.B | 4.C | 4.D | 4.E |
|---|---|---|---|---|---|
| Sodium mycophenolate | 4.810 | 4.810 | 4.810 | 4.810 | 4.810 |
| Povidone K-30 | 0.500 | 0.500 | 0.375 | 0.563 | 0.375 |
| Aerosil 200 | 0.165 | 0.165 | 0.075 | 0.075 | |
| Ethanol 94% for granulation | q.s. | q.s. | q.s. | q.s. | q.s. |
| Hydroxypropyl methyl cellulose | 0.138 | 0.138 | | | |
| Lactose, anhydrous | 1.006 | 1.006 | | | |
| Microcrystalline cellulose | | | 1.377 | 0.940 | 1.015 |
| Starch Sta RX | 0.210 | 0.210 | | | |
| Crospovidone | 0.766 | | 0.250 | | |
| Magnesium stearate | 0.155 | 0.155 | 0.113 | 0.113 | 0.113 |
| Total Core | 7.750 | 6.984 | 7.000 | 6.500 | 6.500 |

TABLE 2
Compositions of a minitablet of sodium mycophenolate (amounts given
in mg)

| Core | 4.F | 4.G | 4:H | 4:I |
|---|---|---|---|---|
| Sodium mycophenolate | 3.103 | 3.103 | 3.103 | 3.103 |
| Povidone (K-30) | 0.323 | 0.323 | 0.323 | 0.323 |
| Silica colloidal anhydrous | 0.106 | 0.106 | 0.106 | 0.106 |
| Ethanol 94%* | q.s. | q.s. | q.s. | q.s. |
| Lactose anhydrous | 0.726 | 0.892 | 0.750 | — |
| Microcrystalline cellulose | — | — | — | 0.750 |
| Maize starch | 0.166 | — | — | — |
| Magnesium stearate | 0.077 | 0.076 | 0.078 | 0.078 |
| Total core | 4.500 | 4.750 | 4.360 | 4.360 |

*removed during processing

TABLE 3
Compositions of a minitablet of mycophenolate mofetil (amounts given in
mg)

| Core | 4.J | 4.K | 4.L | 4.M |
|---|---|---|---|---|
| Mycophenolate mofetil | 4.060 | 4.060 | 4.060 | 4.060 |
| Povidone (K-30) | 0.375 | 0.375 | 0.563 | 0.563 |
| Microcrystalline cellulose | 2.202 | 1.607 | 1.607 | 1.764 |
| Hydroxypropylmethylcellulose 3 cps | — | 0.345 | 0.407 | — |
| Croscarmellose sodium | 0.250 | | | |
| Magnesium stearate | 0.113 | 0.113 | 0.113 | 0.113 |
| Total core weight | 7.000 | 6.500 | 6.750 | 6.500 |

The minitablets containing a core as defined in Tables 1-3 are coated using one of the coating formulations indicated below.

The coated minitablets may be filled into hard gelatine capsules or in stickpacks. For example 60 minitablets having the composition of Table 2 may be filled in a hard gelatine capsule of size 00, or 40 minitablets having the composition of Table 1 or 3 may be filled in a hard gelatine capsule of size 0. All compositions are calculated to give 180 mg mycophenolic acid per capsule, that means per 40 or per 60 minitablets, respectively.

### EXAMPLE 5

### Coating Formulations from Aqueous Dispersions

The coating polymers are dispersed in water to yield an aqueous dispersion. For coating dispersion preparation the antisticking agent is dispersed in water, the plastisizer is dissolved or dispersed, the soluble polymers is dissolved and finally the aqueous polymer dispersion (concentrate=30% polymer) is added. The dispersion is stirred during the coating process.

### EXAMPLE 5.A:

Composition (amounts given in %): the ratio RS:RL is 95:5 up to 70.30, more preferred 90:10 up to 80.20. The polymer is added as 30% aqueous dispersion.

| Eudragit RS30D | 37.5 (polymer: 11.25) | 41.7 (polymer: 12.51) |
|---|---|---|
| Eudragit RL30D | 2.08 (polymer: 0.625) | 4.62 (polymer: 1.39) |
| Triethylcitrate | 2.70 | 2.80 |
| Talc | 6.25 | |
| Syloid 244 | | 4.18 |
| Water | 51.47 | 46.7 |

The preferred amount of the coating dispersion (or suspension) to be sprayed onto the beads, pellets, granules or minitablets are from 10 up to 30%.

### EXAMPLE 5.B

The ratio ethylcellulose:HPMC is 100:0 up to 60:40, more preferred 95:5 up to 80:20.

Composition (amounts given in %)

| | | | |
|---|---|---|---|
| Aquacoat ECD^{R} | 52.78 | 39.55 | 25.11 |
| (dispersion containing about 30% ethylcellulose) | | | |
| Hydroxypropylmethylcellulose | 0.83 | 1.36 | 1.33 |
| Dibutylsebacate or Triethylcitrate | 3.96 | 3.24 | 2.22 |
| Water | 42.43 | 55.85 | 71.33 |

These coating dispersions are preferably applied to an amount of 10-20% based on the total composition weight of minitablet, granule, pellet or layered bead cores.

The coating amount applied to minitablets, pellets, beads, granules is preferably between 5 and 20%.

The ratio ethylcellulose:HPMC AS is 100:0 up to 40:60, more preferred 90:10 up to 60:40.

Composition (amounts given in %)

| | | | | | | |
|---|---|---|---|---|---|---|
| Aquacoat ECD | 39.55 | 20.50 | 18.30 | 17.20 | 16.11 | 13.91 |
| (30% ethylcellulose | | | | | | |
| dispersion) | | | | | | |
| Hydroxypropylmethyl | 1.36 | 1.46 | 2.20 | 2.56 | 2.93 | 3.66 |
| cellulose-acetate succinate | | | | | | |
| (Aqoat AS-MF) | | | | | | |
| (enteric pore former) | | | | | | |
| Triethylcitrate | 3.24 | 1.90 | 1.90 | 1.90 | 1.90 | 1.90 |
| Water | 55.85 | 76.13 | 77.60 | 79.06 | 79.06 | 80.53 |

### EXAMPLE 5.D

| | % |
|---|---|
| Acryl-eze (using Eudragit L100-55; Colorcon) | 99.9 |
| Simethicon 30% (anti-foaming agent) | 0.1 |
| Water | q.s. |

This coating dispersion is preferably applied to an amount of 10-50% based on the total composition weight on drug granules or drug crystals. The desired release profile is yielded by a specific coating weight.

### EXAMPLE 6

### Coating Formulations from Organic Solutions

The coating polymers and plastizisers are dissolved in the organic solvent/solvent mixture. The antisticking agent is finally dispersed in the coating solution

### EXAMPLE 6.A

The coating polymers are dissolved in isopropanol to yield an organic solution. The ratio Eudragit RS:RL is 95:5 up to 70:30. The coating may be applied to minitablets, pellets, granules or layered beads. The preferred amount of coating to be sprayed on the mulitparticulates is from 5 up to 15%

Composition (amounts given in %)

| Eudragit RS 12.5 | 30.93 (polymer: 3.867) |
|---|---|
| Eudragit RL 12.5 | 10.30 (polymer: 1.288) |
| Triethylcitrate | 0.52 |
| Syloid 244 | 1.55 |
| Acetone | 28.35 |
| Isopropanol | 28.35 |

### EXAMPLE 6.B

The coating ingredients are dissolved in ethanol to give a coating solution to be applied on minitablets, beads, pellets and granules. The ratio Ethylcellulose:HPMC is 100:0 up to 50:50, most preferred 95:5-70:30. This coating solution is preferably applied to an amount of 10-15% based on the total composition weight.

Composition (amounts given in %)

| | | | |
|---|---|---|---|
| Ethylcellulose N-010 | 7.00 | 6.75 | 6.38 |
| (polymer) | | | |
| Hydroxypropylmethylcellulose 3 cps | 0.35 | 0.75 | 1.12 |
| (pore former) | | | |
| Aerosil 200 | 1.40 | 1.50 | 1.50 |
| (antisticking agent) | | | |
| Ethanol | 45.63 | 45.50 | 45.50 |
| Aceton | 45.63 | 45.50 | 45.50 |

### EXAMPLE 6.C

The coating ingredients are dissolved in ethanol to give a coating solution, to be applied on minitablets, granules, beads and pellets. The ratio Ethylcellulose:enteric pore former is 100:0 up to 50:50, most preferred 95:5 up to 70:30.

Composition (amounts given in %)

| | | |
|---|---|---|
| Ethylcellulose | 6.75 | 6.00 |
| Hydroxypropylmethylcellulose-phtalate or | 0.75 | 2.50 |
| Hydroxypropylmethylcellulose-acetate-succinate | | |
| (enteric pore former) | | |
| Aerosil 200 (antisticking agent) | 1.50 | 1.50 |
| Ethanol | 45.50 | 45.50 |
| Acetone | 45.50 | 45.50 |

### EXAMPLE 6.D

The coating ingredients are dissolved in ethanol 96% to give a coating solution (to be applied on granules, drug crystals and microparticles).

| | % |
|---|---|
| Ethylcellulose N7 | 83.3 |
| Triethylcitrate | 16.7 |
| Ethanol 96% | q.s. |

This coating solution is preferably applied to an amount of 10-50% based on the total composition weight. The desired release profile is yielded by a specific coating weight.

### EXAMPLE 7

### Enteric Coating to be Applied as Overcoating

The following coating formulations can be applied as overcoating. The coating is applied in an amount of 10-20% of core weight depending on particle (core) size.

### EXAMPLE 7.A

| | | | |
|---|---|---|---|
| Eudragit L 30 D (dry) | 75% | 70% | 75% |
| Triacetine | 7.5% | 10% | 17.5 |
| Syloid 244 FP | 17.5% | 20% | |
| talc | | | 7.5 |
| Water | q.s. | q.s. | q.s. |

### EXAMPLE 7.B

| | | | |
|---|---|---|---|
| HP 50 (dry) | 70% | 74% | 72% |
| Triethylcitrate | 7% | 3% | 7% |
| Colloidal silicon dioxide | 23% | 23% | |
| Talc | | | 21% |
| Acetone, Ethanol 94% 1:1 | q.s. | q.s. | q.s |

Alternatively, an organic solution of Eudragit L100-55 instead of an aqueous dispersion of Eudragit L 30 D may be used in the enteric coating formulations given above.

### EXAMPLE 8

### Coated Multiparticulate Forms

### EXAMPLE 8.A.

### Coated Granules

Granules of formulation 1.B are coated with Coating formulation 6.4 in a Wurster fluid bed equipment until a coating weight of 22% is reached (dry weight of coat as percentage of the uncoated granule weight).

TABLE 4

| Time (min.) | Drug Released (%) | CV % |
|---|---|---|
| 0 | 0 | |
| 15 | 23 | 11 |
| 30 | 35 | 6 |
| 45 | 44 | 4 |
| 60 | 52 | 3 |
| 120 | 71 | 1 |
| 180 | 80 | 1 |
| 240 | 85 | 1 |
| 300 | 89 | 1 |
| 360 | 91 | 1 |
| 480 | 94 | 1 |
| 600 | 96 | 1 |
| 720 | 98 | 1 |
| 960 | 100 | 1 |
| 1200 | 101 | 1 |

Dissolution testing is performed in a paddle apparatus with 50 RPM. The dissolution medium is phosphate buffer pH 6.8.

The granules are coated in a Wurster fluid bed equipment until a coating weight of 30% is reached (dry weight of coat as percentage of the uncoated granule weight).

TABLE 5

| Time (min.) | Drug Released (%) | CV % |
|---|---|---|
| 0 | 0 | |
| 15 | 7 | 8 |
| 30 | 13 | 4 |
| 45 | 18 | 3 |
| 60 | 23 | 3 |
| 120 | 40 | 3 |
| 180 | 52 | 3 |
| 240 | 60 | 3 |
| 300 | 66 | 3 |
| 360 | 70 | 2 |
| 480 | 76 | 2 |
| 600 | 79 | 2 |
| 720 | 82 | 2 |
| 960 | 86 | 1 |
| 1200 | 88 | 1 |

Dissolution testing is performed in a paddle apparatus with 50 RPM. The dissolution medium is phosphate buffer pH 6.8.

### EXAMPLE 9

The granules of formulation 1.B are coated with Coating formulation 5.D in a Wurster fluid bed equipment until a coating weight of 30% is reached (dry weight of coat as percentage of the uncoated granule weight)

TABLE 6

| Time (min.) | Drug Released (%) |
|---|---|
| 0 | 0 |
| 15 | 1 |
| 30 | 2 |
| 45 | 3 |
| 60 | 4 |
| 75 | 4 |
| 90 | 5 |
| 105 | 5 |
| 120 | 6 |
| 135 | 83 |
| 150 | 97 |
| 165 | 99 |
| 180 | 99 |

Dissolution testing is performed in a paddle apparatus with 50 RPM. The dissolution medium is 750 ml of hydrochloric acid pH 1 (first 2 h) and then added 250 mg sodium phosphate solution to increase the pH to 6.8.

This formulation meets the specifications for delayed release.

### EXAMPLE 10

### Coated Minitablets

To compare the influence of the amount of enteric pore former (HPMC-AS) used in the ethylcellulose diffusion coat of the minitablet core formulation 4.B coated with coating formulation 5.C variants. The following dissolution method is chosen: pH 6.8 phosphate buffer (0.05M) 1000 ml, Paddle 50 rpm.

The drug release in % over time is indicated in the table below:

TABLE 7

| | M (20% Aqoat) | | N (30% Aqoat) | | Q (35% Aqoat) | | P (40% Aqoat) | | R (50% Aqoat) | |
|---|---|---|---|---|---|---|---|---|---|---|
| | (n = 2) | | (n = 2) | | (n = 3) | | (n = 3) | | (n = 3) | |
| Time (min) | DR % | srel % | DR % | srel % | DR % | srel % | DR % | srel % | DR % | srel % |
| 0 | 0 | 0 | 0 | 0 | 0 | 0 | 0 | 0 | 0 | 0 |
| 30 | 2.2 | 101.4 | 1.6 | 81.4 | 4.7 | 7.9 | 12.1 | 12.0 | 31.4 | 4.4 |
| 60 | 5.1 | 101.8 | 5.1 | 52.6 | 22.3 | 2.2 | 36.6 | 7.8 | 76.7 | 1.1 |
| 120 | 15.2 | 58.8 | 19.0 | 20.4 | 57.6 | 1.0 | 76.9 | 2.2 | 96.5 | 0.5 |
| 180 | 26.7 | 38.1 | 34.0 | 13.0 | 78.0 | 0.3 | 91.2 | 1.0 | 98.9 | 0.5 |
| 240 | 37.2 | 27.4 | 47.2 | 9.8 | 87.8 | 0.3 | 96.0 | 0.6 | 99.8 | 0.6 |
| 300 | 46.3 | 21.4 | 57.0 | 7.6 | 92.6 | 0.4 | 98.2 | 0.8 | — | — |
| 360 | 53.4 | 17.6 | 64.6 | 6.1 | 95.1 | 0.5 | 99.3 | 0.4 | — | — |
| 480 | 63.5 | 12.9 | 76.3 | 3.8 | 97.4 | 0.6 | 100.3 | 0.5 | — | — |

### Effect of Acidic Pretreatment:

The dissolution profiles of the minitablet formulation 4.B coated with coating formulation 5.B (Aquacoat+10% HPMC) and minitablet formulation 4.B coated with coating formulation 5.C (Aquacoat+10% HPMC-AS) with acidic pretreatment (first 2 h at pH 1 than buffered to pH 6.8) and without acidic pretreatment (only at pH 6.8) indicate the acidic sensitivity of HPMC as pore former compared to the significantly reduced acidic sensitivity of HPMC-AS as pore former. The drug release over time is shown in the table below applying the following dissolution methods:

Dissolution testing is performed in a paddle apparatus with 50 RPM. The dissolution medium is 750 ml of hydrochloric acid pH 1 (first 2 h) and then added 250 mg sodium phosphate solution to increase the pH to 6.8. In both cases no drug is released in acidic medium due to low solubility of mycophenolic acid in acidic medium, but the film with the soluble pore former HPMC shows swelling of the film coating and formation of free mycophenolic acid during 2 hours pretreatment in acidic medium what affects the drug release in buffer pH 6.8.

The dissolution rate profile is strongly affected by acidic pretreatment for HPMC as pore former, while the dissolution rate profile is less affected by acidic pretreatment using the enteric polymer HPMC AS as pore former.

TABLE 8

| | | | | | 10% | | 10% | |
|---|---|---|---|---|---|---|---|---|
| | 10% HPMC | | 10% HPMC | | HPMC AS | | HPMC AS | |
| Time (min) | pH 6.8 | | pH 1 and 6.8 | | pH 6.8 | | pH 1 and 6.8 | |
| (n = 3) | DR % | srel % | DR % | srel % | DR % | srel % | DR % | srel % |
| 0 | 0 | 0 | 0.9 | 7.6 | 0 | 0 | 4.3 | 7.4 |
| 30 | 18.6 | 1.4 | 5.0 | 2.5 | 16.6 | 2.0 | 37.7 | 19.3 |
| 60 | 38.9 | 1.6 | 8.9 | 0.7 | 33.4 | 1.7 | 51.2 | 17.4 |
| 120 | 64.1 | 1.1 | 20.3 | 2.0 | 58.4 | 1.2 | 67.5 | 12.8 |
| 180 | 75.5 | 1.0 | 31.6 | 2.2 | 72.5 | 1.0 | 77.0 | 9.5 |
| 240 | 82.2 | 0.9 | 40.9 | 1.9 | 80.6 | 0.8 | 83.0 | 6.9 |
| 300 | 86.8 | 0.7 | 48.5 | 1.5 | 85.9 | 0.6 | 87.0 | 5.1 |
| 360 | 90.1 | 0.7 | 54.7 | 1.3 | 89.3 | 0.5 | 89.8 | 3.8 |
| 480 | 94.1 | 0.8 | 63.6 | 1.2 | 93.4 | 0.5 | 93.3 | 2.1 |

### EXAMPLE 11

### Multiparticulates Coated by Coat Precipitation

A polymer solution is firstly prepared by dissolving the ethylcellulose and the polyethylene in cyclohexane with heating and stirring. Subsequently, the substance drug and the stabilizer are added and the dispersion allowed to cool whilst stirring. The resultant coated microparticles are washed and dried and could be further coated with one of the coating formulations below.

Coated drug particles may then be formulated into a capsule or sachet by the addition of bulking agents and lubricants or further compressed into tablets or minitablets.

Composition of the core (amounts given in %)

| | | | |
|---|---|---|---|
| MPA, Na Mycophenolate or MMF | 74% | 79% | 84% |
| Ethylcellulose | 21% | 16% | 11% |
| Polyethylene | 1% | 1% | 1% |
| Colloidal silica (Syloid ®) | 4% | 4% | 4% |
| Cyclohexane | qs* | qs* | qs* |

*Not part of the formulation.

### EXAMPLE 12

### Tablet Formulation from Modified Release Pellets (i.e. Formulation 3.A

Modified release coated pellets are mixed with the other ingredients and compressed on a rotary tablet press into tablets (one 834 mg oblong tablet corresponds to 180 mg mycophenolic acid).

TABLE 9

| | | | |
|---|---|---|---|
| Modified release coated | 50% | | |
| pellets | | | |
| Sodium mycophenolate | (23.2%) | 192.4 | (60% of the pellet) |
| Pellet core excipients | (15.4%) | 128.3 | |
| Pellet coating | (11.5%) | 96.2 | |
| MCC (Avicel pH 101) | 22% | 183.5 | |
| Avicel granulate | 21% | 175.3 | |
| Crospovidone | 6% | 50.0 | |
| Magnesium stearate | 1% | 8.3 | |
| Total | 100% | 834.0 | |

### EXAMPLE 13

### Tablet Formulation from Modified Release Granules (Formulation 1.B with Coating Formulation 6.D

### EXAMPLE 13.A

Mixtures of coated granules with 22% coat weight corresponding mycophenolic acid and excipients (30% by total tablet weight) are blended in a bag and the amount of mixture for one tablet (380 mg) is weighed and filled into the die and compressed on an excentric tablet press (Korsch EK0) using 10 mm round shaped punches. The tablets are evaluated for hardness, disintegration, friability and dissolution rate.

TABLE 10

| | | | hardness | Disintegration | Friability |
|---|---|---|---|---|---|
| Composition | mg | % | (kP) | time (min′sec″) | (%) |
| Mycophenolate | 264 | 70 | 9-9.5 | 5′03-5′18″ | 0.13 |
| coated granulat | | | | | |
| Ludipress | 92 | 24 | | | |
| LHPC LH11 | 23 | 6 | | | |
| Magnesium | 1 | 0.25 | | | |
| stearate | | | | | |

### EXAMPLE 13.B

TABLE 11

| | | | hardness | Disintegration | Friability |
|---|---|---|---|---|---|
| Composition | mg | % | (kP) | time (min′sec″) | (%) |
| Mycophenolate | 264 | 70 | 10-12 | 1′20″-2′50″ | 0.00 |
| coated granulat | | | | | |
| Microcrystalline | 92 | 24 | | | |
| cellulose (e.g. | | | | | |
| Avicel PH200) | | | | | |
| LHPC LH11 | 23 | 6 | | | |
| Magnesium | 1 | 0.25 | | | |
| stearate | | | | | |

Dissolution Results of Tablets with Coated Granules

Dissolution testing is performed in a paddle apparatus with 50 RPM. The dissolution medium is 750 ml of hydrochloric acid pH 1 (first 2 h) and then added 250 mg sodium phosphate solution to increase the pH to 6.8.

TABLE 12

| | Drug released % | Drug released % | |
|---|---|---|---|
| | (Coated granules | (Tablets | Drug released % |
| Time | (Formulation 1.B; | Formulation | (Tablets Formulation |
| (min) | coat formulation 6.D) | 13A) | 13.B) |
| 0 | 0 | 0 | 0 |
| 30 | 3 | 4 | 5 |
| 60 | 6 | 7 | 6 |
| 120 | 11 | 12 | 8 |
| 180 | 66 | 68 | 54 |
| 240 | 75 | 79 | 70 |
| 300 | 81 | 84 | 78 |
| 420 | 87 | 89 | 87 |
| 540 | 91 | 93 | 92 |
| 600 | 92 | 94 | 94 |
| 720 | 94 | 96 | 96 |
| 960 | 97 | 98 | 99 |
| 1200 | 99 | 99 | 100 |

A high load of coated granules in the tablet is achieved. Tablets are measured with standard In-Process-Control tests and do show sufficient results. Therefore compompaction forces applied do not significantly alter the dissolution profile compared to the one of the coated granules used for the tablet production.

### EXAMPLE 14

### Tablet Formulation from Modified Release Granules

Mixtures of coated granules (Formulation 1.B with coat formulation 6.D) with 22% coat weight corresponding to a dose 180 mg of mycophenolic acid (MPA) and excipients (30% by total tablet weight) are blended in a bag and the suitable amount of mixture for one tablet (760 mg for 360 mg MFA or 1520 mg for 720 mg MFA) is weighed and filled into the die and compressed on an excentric tablet press (Korsch EK0) using 19*8 mm (for 360 mg MPA) or 22*11 mm (for 720 mg MPA) capsule shaped punches. Tablets are evaluated for hardness, disintegration, friability and dissolution rate.

### EXAMPLE 14.A

TABLE 13
Tablet 19 * 8 mm

| | | | Hardness | Disintegration | Friability |
|---|---|---|---|---|---|
| Composition | mg | % | (kP) | time (min′sec″) | (%) |
| Mycophenolate | 528 | 70 | 15.5-16 | 1′05″-1′30″ | 0 |
| coated granulat | | | | | |
| Microcrystalline | 184 | 24 | | | |
| cellulose | | | | | |
| (e.g. Avicel | | | | | |
| PH200) | | | | | |
| LHPC LH11 | 46 | 6 | | | |
| Magnesium | 2 | 0.25 | | | |
| stearate | | | | | |

### EXAMPLE 14.B

TABLE 14
Tablet (22 * 11 mm)

| | | | Hardness | Disintegration | Friability |
|---|---|---|---|---|---|
| Composition | mg | % | (kP) | time (min′sec″) | (%) |
| Mycophenolate | 1056 | 70 | 20-21 | 1′32″-1′34″ | 0.02 |
| coated granulat | | | | | |
| Microcrystalline | 368 | 24 | | | |
| cellulose (e.g. | | | | | |
| Avicel | | | | | |
| PH200) | | | | | |
| LHPC LH11 | 92 | 6 | | | |
| Magnesium | 4 | 0.25 | | | |
| stearate | | | | | |

### Dissolution Results

Dissolution testing is performed in a paddle apparatus with 50 RPM. The dissolution medium is phosphate buffer pH 6.8.

TABLE 15

| | Drug released % | | |
|---|---|---|---|
| | (Coated granules | Drug released % | |
| | (Formulation 1.B; | (Tablets | Drug released % |
| Time | coat formulation 6.D, | Formulation | (Tablets Formulation |
| (min) | 22% coat weight) | 14.A 19 * 8 mm) | 14.B 22 * 11 mm) |
| 0 | 0 | 0 | 0 |
| 30 | 35 | 34 | 30 |
| 60 | 51 | 52 | 45 |
| 120 | 70 | 72 | 64 |
| 180 | 79 | 81 | 76 |
| 240 | 84 | 86 | 82 |
| 300 | 87 | 89 | 87 |
| 420 | 90 | 91 | 90 |
| 540 | 94 | 95 | 95 |
| 600 | 95 | 96 | 95 |
| 720 | 97 | 97 | 97 |
| 960 | 98 | 99 | 99 |
| 1200 | 100 | 100 | 100 |

A high load of coated granules in the tablet is achieved. Tablets are measured with standard In-Process-Control tests and do show sufficient results. Therefore compompaction forces applied do not significantly alter the dissolution profile compared to the one of the coated granules used for the tablet production.

### EXAMPLE 15

### Preparation of Coated/Embedded Drug Microparticles

A Na Mycophenolate suspension of the desired particle size range is prepared by high pressure homogenization in Acetone/Ethanol 50/50% with addition of small amount (<5%) of polymer (e.g. Ethylcellulose) for stabilization purposes.

After achieving the correct particle size distribution, more ethylcellulose is dissolved in the homogenized drug suspension under stirring. Subsequently, this suspension is spray dried to form polymer-coated crystalline drug particles or drug particles embedded in a polymer matrix, depending on the drug/polymer ratio. The resultant coated microparticles could be further coated with one of the coating formulations below.

Coated drug particles may then be formulated into a capsule or sachet by the addition of bulking agents and lubricants or further compressed into tablets or minitablets.

Composition (amounts given in %) of the Core

| | | |
|---|---|---|
| MPA, Na Mycophenolate or MMF | 20% | 80% |
| Ethylcellulose | 80% | 20% |
| Acetone/Ethanol | qs* | qs* |

*Not part of the formulation

1. A composition comprising mycophenolic acid, a salt or a prodrug thereof in a modified release form.

2. A composition according to claim 1 which is a tablet, a capsule or in the form of multiparticulates and comprises a modified release coating.

3. A composition according to claim 2 which is in the form of multiparticulates which are compressed into tablet or dispensed in a capsule or a sachet.

4. A composition according to claim 3 wherein the multiparticulates comprise a modified release coating.

5. A composition according to claim 3 wherein the tablet or capsule is able to disintegrate or dissolve in the mouth, stomach or small intestine to give modified release coated multiparticulates.

6. A composition according to claim 2 wherein the composition comprise a mixture of multiparticulates which provide different modified release profiles.

7. A composition according to claim 2 wherein the multiparticulates are microparticles, minitablets, pellets, granules, beads or drug particles.

8. A composition according to claim 2 wherein the modified release coating is a diffusion coating.

9. A composition according to claim 2 wherein the coating comprises cellulose derivative, acrylic copolymer, methacrylic copolymer or mixture thereof.

10. A composition according to claim 9 wherein the coating comprises ethylcellulose, acrylic or methacrylic copolymer containing quaternary ammonium groups or mixture thereof.

11. A composition according to claim 10 wherein the acrylic or methacrylic copolymer contains tri(C₁₋₄alkyl)-ammonium methylmethacrylate groups.

12. A composition according to claim 1 wherein the composition comprises one or more excipients selected from a plasticizer, an antisticking agent, a wetting agent, a thickening agent and a pore former, preferably a pH-dependent pore former.

13. A composition according to claim 2 wherein the coating comprises a pH-dependent pore former selected from the group consisting of hydroxypropylmethylcellulose-phthalate, hydroxypropylmethylcellulose-acetate-succinate, methylacrylic acid copolymer, ethylacylic acid copolymer, celluloseacetatephthalate, polyvinylacetatephthalate and mixture thereof.

14. A composition according to claim 2 wherein the coating comprises a pore former selected from the group consisting of polyethyleneglycol, polyvinylpyrrolidone, polyethyleneoxide, cellulose derivative, hydroxypropylmethylcellulose, hydroxypropylcellulose, water-soluble acrylate esters, water-soluble acrylate methacrylate esters, polyacrylic acid, PEG, triacetine, triethylcitrate, hydrophilic silicium dioxide and mixture thereof.

15. A composition according to claim 2 wherein an enteric coating is coated upon the modified release coating.

16. A composition according to claim 15 wherein the enteric coating comprises cellulose acetate phthalate, cellulose acetate trimellitate, methacrylic acid copolymers, hydroxypropyl methylcellulose phthalate, hydroxypropylmethylcellulose acetate succinate, polyvinylacetatephthalate cellulose acetate phthalate; cellulose acetate trimellitate; methacrylic acid copolymers, e.g. copolymers derived from methylacrylic acid and esters thereof, containing at least 40% methylacrylic acid; hydroxypropyl methylcellulose phthalate; hydroxypropylmethylcellulose acetate succinate or Polyvinylacetatephthalate, mixture thereof.

17. A composition according to claim 1 containing mycophenolic acid, mycophenolate mofetil or sodium mycophenolate.

18. A fixed combination comprising a) a composition according to claim 1 and b) rapamycin or a rapamycin derivative.

19. A composition according to claim 1 for use in the treatment or prevention of native or transgenic organ, tissue or cellular allograft or xenograft transplant rejection, or treatment or prevention of immune-mediated and/or inflammatory disease.

20. A method of immunosuppressing a subject which comprises administering a composition according to claim 1 to a subject in need thereof, optionally with the simultaneous, sequential or separate administration of another immunosuppressant.

21. A method for reducing inter- and intrapatient PK variability in a subject comprising administering a therapeutically effective amount of a composition according to claim 1.
